(19) United States
(12) Patent Application Publication
(10) Pub. No.: US 2026/0335288 A1
(43) Pub. Date: Oct. 29, 2026

(54) Pharmacologic Targeting of Genetic Factors That Control Jet Lag

(71) Applicants: University of Pittsburgh-Of the Commonwealth System of Higher Education, Pittsburgh, PA (US); The Research Foundation for the State University of New York, Albany, NY (US)

(72) Inventors: Stephen Yu-Wah Chan, Pittsburgh, PA (US); Colleen A. McClung, Gibsonia, PA (US); Ivet Bahar, Pittsburgh, PA (US); Wei Sun, Sewickley, PA (US); Hongying Cheng, Pittsburgh, PA (US)

(21) Appl. No.: 19/107,706

(22) PCT Filed: Sep. 1, 2023
(86) PCT No.: PCT/US2023/031889

§ 371 Date: Feb. 28, 2025

Related U.S. Application Data
(60) Provisional application No. 63/374,456, filed on Sep. 2, 2022.

(51) Int. Cl. A61K 31/4162; C07D 487/20

(52) U.S. Cl. CPC A61K 31/4162; C07D 487/20

(57) ABSTRACT

Methods, compositions, and devices are provided for use in controlling jet lag, chronotype, and/or circadian rhythm in a patient. Patients can be treated with a compound that binds NPL, to control the patient's jet lag, chronotype, and/or circadian rhythm.

### CROSS REFERENCE TO RELATED APPLICATIONS

This application claims priority to U.S. Provisional Patent Application No. 63/374,456 filed Sep. 2, 2022, the disclosure of which is hereby incorporated herein by reference in its entirety.

Jet lag remains a significant problem for individuals who frequently travel or work variable night and day shifts. Jet lag is caused by a misalignment between an individual's internal, biologically driven circadian clock and the environmental light/dark cycle. There are limited options to counteract jet lag, and current treatments are not the strongest synchronizers of rhythms, they take time to work, and they don't work equally well for everyone. Moreover, we have limited understanding of why some individuals are particularly susceptible to jet lag while others seem to be relatively spared. The chronotype of an individual (e.g., being a “morning person” or “night owl”) may be an important determinant of this susceptibility. Undoubtedly there is a strong genetic component to this vulnerability. However, our knowledge of the gene variations that lead to chronotype differences, and mechanisms that control them, is extremely limited. Fortunately, recent advances in the study of human genetics and genomics have paved the way for studies of the genetic susceptibility to jet lag and an inability to adapt to altered day/night cycles. Moreover, we can use this knowledge of what makes someone vulnerable, to help design customized treatments.

Methods of controlling or treating jet lag, a patient's chronotype, or a patient's circadian rhythm are desired.

### SUMMARY

A method of reducing jet lag, modifying chronotype, and/or or modulating a circadian clock of a patient, e.g., a human patient, is provided herein, comprising: administering to the patient a compound that binds NPL, RGS16, MYH9, and/or MYH10 and/or modulates interaction of MYH9 and/or MYH10 with an endogenous nucleic acid sequence comprising rs12736689 in an amount effective to reduce jet lag or to modulate the circadian clock of the patient.

A compound is provided, having the structure (i):

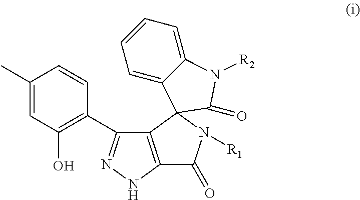

wherein, R₁ and R₂, are, independently, methyl, hydroxyl, or hydrogen, and R₁ and R₂ are not both methyl, or a pharmaceutically-acceptable salt thereof.

A composition also is provided herein, comprising: a compound having the structure (i):

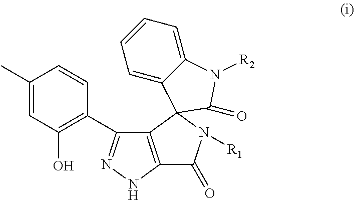

wherein, R₁ and R₂, are, independently, methyl, hydroxyl, or hydrogen, or a pharmaceutically-acceptable salt thereof, in an amount effective to reduce jet lag, modify chronotype, and/or modulate a circadian clock of a patient, e.g., a human patient; and a pharmaceutically-acceptable excipient.

The following numbered clauses outline various non-limiting aspects and embodiments of the invention.

Clause 1. A method of reducing jet lag, modifying chronotype, and/or or modulating a circadian clock of a patient, e.g., a human patient, comprising: administering to the patient a compound that binds NPL, RGS16, MYH9, and MYH10 and/or prevents interaction of MYH9 and/or MYH10 with an endogenous nucleic acid sequence comprising rs12736689 in an amount effective to reduce jet lag or to modulate the circadian clock of the patient.

Clause 2. The method of clause 1, comprising administering a compound that binds NPL to the patient in an amount effective to reduce jet lag or to modulate the circadian clock of the patient.

Clause 3. The method of clause 2, wherein the compound that binds NPL is selected from the group consisting of: ZINC4096282, sialic acid alditol, MolPort-005-972-726, MolPort-005-975-059, MolPort-000-523-173, MolPort-004-939-783, ZINC27757562, ZINC102736687, ZINC8845752, ZINC8845753, ZINC68601727, ZINC102738233, ZINC67909210, ZINC4476643, ZINC4595517, ZINC1704299626, MolPort-003-875-380, MolPort-016-586-217 (ZINC4595517), or a pharmaceutically acceptable salt thereof.

Clause 4. The method of clause 2, wherein the compound has the structure

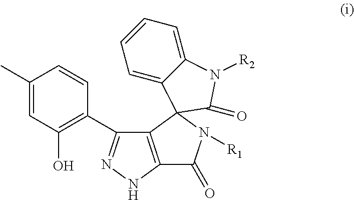

wherein,
   R₁ and R₂, are, independently, methyl, hydroxyl, or hydrogen, or a pharmaceutically-acceptable salt thereof.

Clause 5. The method of clause 4, wherein R₁ is Me or H.

Clause 6. The method of clause 5, wherein R₁ is H.

Clause 7. The method of any one of clauses 4-6, wherein R₂ is Me or OH.

Clause 8. The method of clause 7, wherein R₂ is H.

Clause 9. The method of clause 4, wherein R₁ is H and R₂ is OH.

Clause 10. The method of clause 2, wherein the compound has the structure (ii):

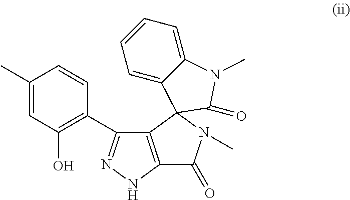

or a pharmaceutically-acceptable salt thereof, e.g., compound 173 (MolPort 000-523-173).

Clause 11. A compound having the structure (i):

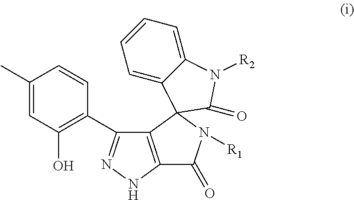

wherein,
   R₁ and R₂, are, independently, methyl, hydroxyl, or hydrogen, and R₁ and R₂ are not both methyl,
or a pharmaceutically-acceptable salt thereof.

Clause 12. The compound of clause 11, wherein R₁ is Me or H.

Clause 13. The compound of clause 12, wherein R₁ is H.

Clause 14. The compound of any one of clauses 11-13, wherein R₂ is Me or OH.

Clause 15. The compound of clause 14, wherein R₂ is H.

Clause 16. The compound of clause 11, wherein R₁ is H and R₂ is OH.

Clause 17. The compound of clause 11, wherein the compound has the structure (ii):

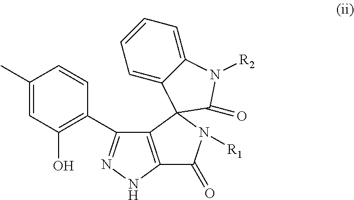

or a pharmaceutically-acceptable salt thereof, e.g., compound 173 (MolPort 000-523-173).

Clause 18. A composition comprising:
a compound having the structure (i):

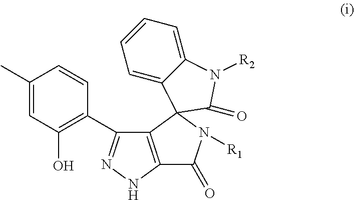

wherein,
   R₁ and R₂, are, independently, methyl, hydroxyl, or hydrogen,
   or a pharmaceutically-acceptable salt thereof in an amount effective to reduce jet lag, modify chronotype, and/or modulate a circadian clock of a patient, e.g., a human patient; and
a pharmaceutically-acceptable excipient.

Clause 19. The composition of clause 18, wherein R₁ and R₂ of the compound are not both methyl.

Clause 20. The composition of clause 18, wherein R₁ of the compound Me or H.

Clause 21. The composition of clause 20, wherein R₁ of the compound is H.

Clause 22. The composition of any one of clauses 18-21, wherein R₂ of the compound is Me or OH.

Clause 23. The composition of clause 22, wherein R₂ of the compound is H.

Clause 24. The composition of clause 18, wherein R₁ of the compound is H and R₂ of the compound is OH.

Clause 25. The composition of clause 18, wherein the compound has the structure (ii):

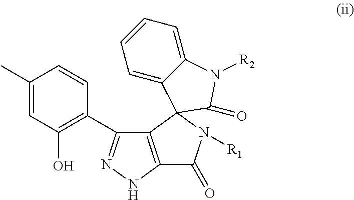

or a pharmaceutically-acceptable salt thereof, e.g., compound 173 (MolPort 000-523-173).

Clause 26. The composition of any one of clauses 18-25 in the form of a parenteral, topical, oral, transdermal, or transmucosal dosage form.

### BRIEF DESCRIPTION OF THE DRAWINGS

FIG. 1: Computation pipeline for virtual screening NPL inhibitors. (A) Pharmacophore model. (B) Top 10 NPL inhibitors predicted by virtual screening of compounds from MolPort. Compounds were ranked based on their computed binding affinities (column Score). (C) Summary of docking simulation results on the top predicted NPL inhibitors. Simulations were performed using Vina. (D) Re-ranking of top inhibitors based on docking occupancy and binding affinity predicted by docking simulations. (E) Predicted binding pose of MolPort-000-523-173 (compound 173) to human NPL.

FIG. 2: Identification of generic structure of NPL inhibitors. (A) Molecular dynamics simulations-resolved binding of compound 173 to human NPL. (B) Generic structure of NPL inhibitor derived from compound 173, where —CH₃ at the R₁ and R₂ positions in compound 173 are replaced by —H and —OH groups, to enhance the interactions with Y143 and T51-T52. (C) Example of a compound 173 analog.

FIGS. 3A-3C provide exemplary structures of compounds described herein.

FIG. 4: Compound 173 phenocopies siNPL in reducing circadian periodicity. Treatment with compound 173 significantly decreased cellular period in an in vitro model of rhythm.

FIG. 5. Graphs showing that compound 173 phenocopies siNPL. Apoptosis (A & D) was increased, and proliferation (B & E) was decreased in human pulmonary artery endothelial cells (PAECs) treated with siNPL or compound 173. Circadian period length was decreased in MF-Luc cells treated with siNPL or compound 173 (C & F). A.U.: arbitrary units.

### DETAILED DESCRIPTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more. Patent publications cited below are hereby incorporated herein by reference in their entirety to the extent of their technical disclosure and consistency with the present specification.

As used herein, the terms “comprising,” “comprise” or “comprised,” and variations thereof, are open ended and do not exclude the presence of other elements not identified. In contrast, the term “consisting of” and variations thereof is intended to be closed and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” of a patient for a condition, such as controlling jet lag, modifying chronotype, and/or modulating a circadian clock of a patient, e.g., a human patient, means administration to a patient by any suitable dosage regimen, procedure and/or administration route of a composition, device, or structure with the object of achieving a desirable clinical/medical end-point, including but not limited to reducing jet lag and associated symptoms, as well as symptoms of other chronotype or circadian rhythm disorders. An amount of any reagent or therapeutic agent, administered by any suitable route, effective to treat a patient is an amount capable of controlling jet lag, modifying chronotype, and/or or modulating a circadian clock of a patient, e.g., a human patient. The therapeutically-effective amount of each therapeutic may range from 1 μg per dose to 10 g per dose, including any amount there between, such as, without limitation, 1 ng, 1 μg, 1 mg, 10 mg, 100 mg, or 1 g per dose. The therapeutic agent may be administered by any effective route, and, for example, as a single dose or bolus, at regular or irregular intervals, in amounts and intervals as dictated by any clinical parameter of a patient, or continuously.

Active ingredients, such as the compounds described below, exemplified by the described 173 compound, may be compounded or otherwise manufactured into a suitable composition for use, such as a pharmaceutical dosage form or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

Useful dosage forms include, for example and without limitation: parenteral, intravenous, intramuscular, intraocular, or intraperitoneal solutions, oral tablets or liquids, topical drops, ointments, or creams, and transdermal devices (e.g., patches). The compound may be a sterile solution comprising the active ingredient (drug or compound), and a solvent, such as water, saline, lactated Ringer's solution, or phosphate-buffered saline (PBS). Additional excipients, such as polyethylene glycol, rheology modifiers, emulsifiers, salts and buffers may be included in the solution.

Suitable dosage forms may include single-dose, or multiple-dose vials or other containers, such as medical syringes or droppers, e.g., eye droppers, containing a composition comprising an active ingredient useful for controlling jet lag, modifying chronotype, and/or modulating a circadian clock of a patient, e.g., a human patient, as described herein.

Pharmaceutical formulations adapted for administration include aqueous and non-aqueous sterile solutions which may contain, in addition to the active pharmaceutical ingredient or drug, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules and tablets.

Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Pharmaceutically acceptable salts of any of the compounds described herein also may be used in the methods described herein. Pharmaceutically acceptable salt forms of the compounds described herein may be prepared by conventional methods known in the pharmaceutical arts, and include as a class veterinarily acceptable salts. For example and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide, and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (drug) comprising a salt form of any compound as described herein. The salt form may confer improved and/or desirable pharmacokinetic/pharmodynamic properties of the compounds described herein.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An amount effective to control jet lag, modify chronotype, and/or or modulate a circadian clock of a patient, e.g., a human patient, may be 1 μg to 10 g, or from 1 ng to 100 mg/kg of compound 173 per day, for example an amount to produce 20 μM±99%, 90%, 80%, 70%, 60%, 50%, 40%, 30%, 25%, 20%, 15%, 10%, 0.5%, 1%, e.g., from 1 to 40 μM, or any increment therebetween of the compound in a patient's bodily fluid, e.g., blood, serum, plasma, etc. Safe and effective amounts of other compounds described herein may be administered.

The compositions and methods described herein are useful to control jet lag, modify chronotype, and/or or modulate a circadian clock of a patient, e.g., a human patient. “Jet lag” or “jet lag disorder” refers to a temporary sleep disorder resulting from quick traversal of one or more time zones, such that the patient's internal clock or circadian rhythm are disrupted because the patient's internal clock is synced to their original time zone and has not yet changed to the time zone of where the patient has traveled. The more time zones crossed, the more likely a patient will suffer from jet lag, and the longer it will last. Jet lag can cause daytime fatigue, cognitive difficulties, an unwell feeling, trouble staying alert, and stomach problems. Although the symptoms are temporary, they can affect a patient's comfort while on vacation or during a business trip. The more often a patient travels between time zones the greater that patient can suffer from jet lag. Dehydration and airline cabin pressure may also contribute to jet lag. Age also can contribute to jet lag, with older patients often needing more time to recover from jet lag.

“Chronotype” or “diurnal preference” is a part of circadian rhythmicity and may be defined as individual variation in the preferred timing of the sleep-wake cycle. It reflects individual variability in the phase of entrainment and is associated with variations of physiological nature, such as the rhythm of body temperature and hormone secretion. Chronotype may evaluated using self-reported questionnaires, usually the Morningness-Eveningness Questionnaire (MEQ), its reduced five-item version (rMEQ), and the Munich Chronotype Questionnaire (MCTQ). The Composite Scale of Morningness (CSM), the Diurnal Type Scale (DTS), the Circadian Type Questionnaire (CTQ), and the Preference Scale (PS) can also be considered instruments for evaluating chronotype. Other tools that may be used to evaluate chronotypes, include, without limitation, core body temperature, dim light melatonin onset (DLMO), a sleep diary, wrist accelerometry, and actigraphs. Morning chronotype individuals, also known as early chronotypes or larks, prefer to be active in the morning and sleep and wake early. Evening chronotypes, also called late chronotypes or owls, prefer to be active in the evening and sleep and wake up late. The intermediate chronotype (neutral or neither type) has no preference for morning or evening. Morning chronotype individuals achieve peak physical and mental performance in the early part of the day after waking up. Conversely, evening chronotype individuals have the best mental and physical performance before sleeping (Zou H, et al. Chronotype, circadian rhythm, and psychiatric disorders: Recent evidence and potential mechanisms. Front Neurosci. 2022 Aug. 10; 16:811771).

Neuraminidase, N-Acetylneuraminic Acid Lyases (NPLs) regulate cellular concentrations of N-acetyl-neuraminic acid (sialic acid) by mediating the reversible conversion of sialic acid into N-acetylmannosamine and pyruvate. An example of NPL includes human NPL, Gene ID: 80896 (NPL N-acetylneuraminate pyruvate lyase [Homo sapiens (human)], also NCBI Reference Sequence: NP_001186979.1 (N-acetylneuraminate lyase isoform 2 [Homo sapiens], for example), and also UniProt Q9BXD5).

As described above, provided herein are methods of reducing or controlling jet lag, modifying chronotype, and/or modulating a circadian clock of a patient, e.g., a human patient, comprising administering an NPL-binding compound to the patient in an amount effective to reduce or control jet lag, modify chronotype, and/or modulate a circadian clock of the patient. The composition may be a parenteral, transdermal, topical, transmucosal, or oral dosage form, comprising the active agent with suitable excipients to form a useful dosage form for delivery to the active agent compound.

An NPL-binding compound may be delivered parenterally, e.g. by injection or infusion, transdermally, transmucosally, or orally. Suitable liquid compositions for delivery include the described drug product as active agent as well as, for example and without limitation: water, rheology modifiers, preservatives, salts, buffers, emulsifiers, and penetration enhancers. A person of ordinary skill in the pharmaceutical and compounding arts can readily ascertain an optimal composition for delivery of the compounds as described herein.

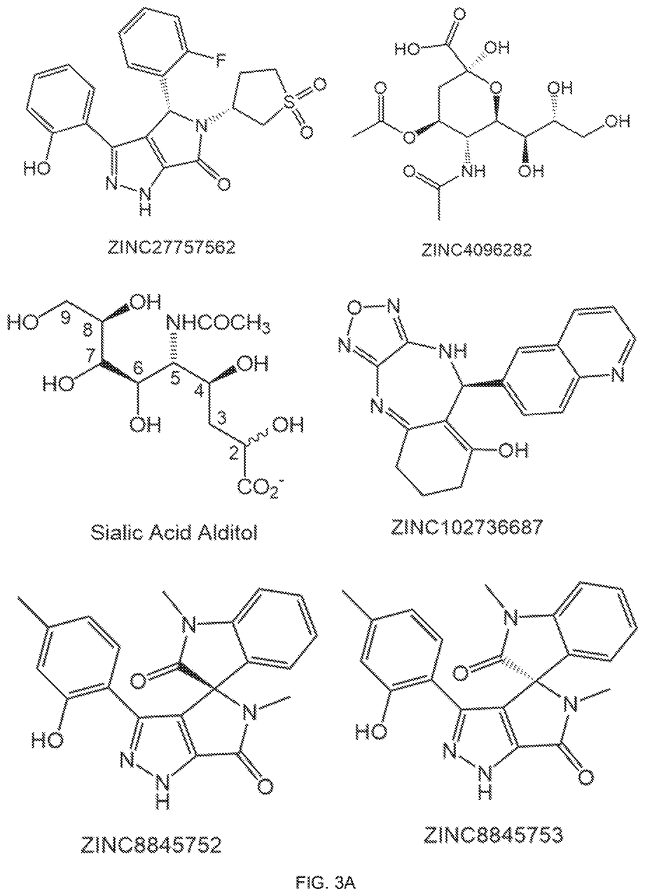

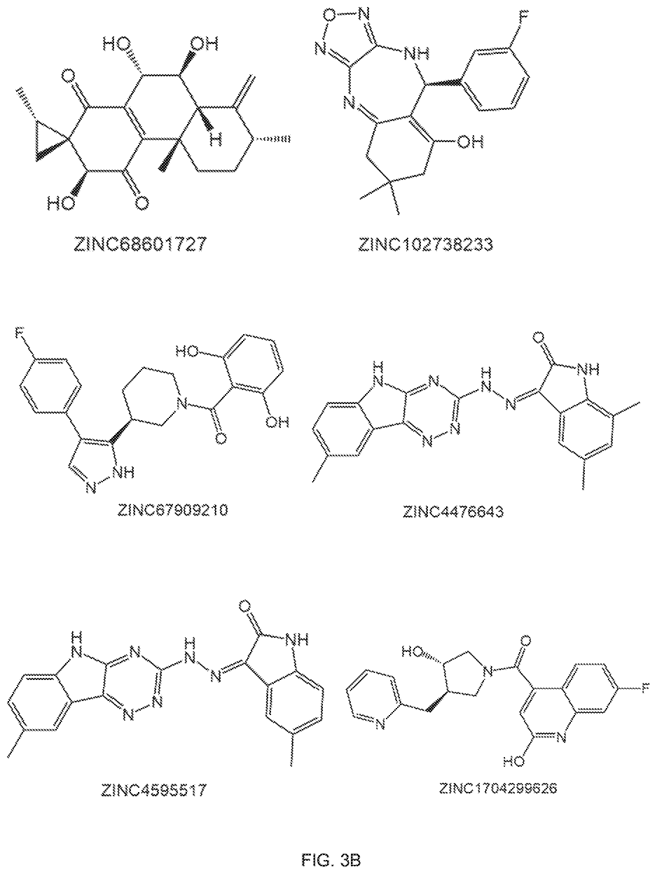

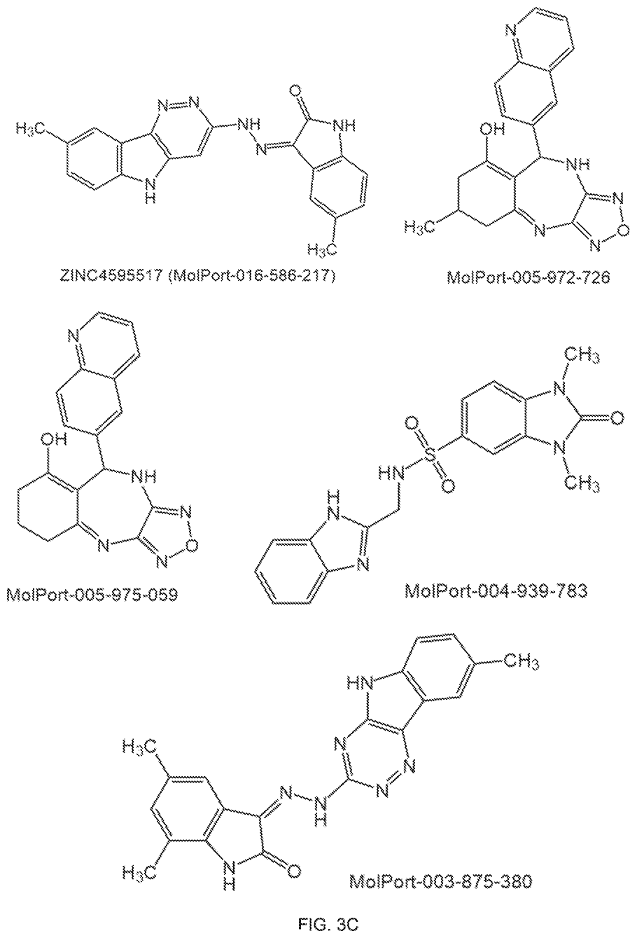

As above, an NPL-binding compound may be administered to reduce or control jet lag, modify chronotype, and/or modulate a circadian clock of a patient, such as a human patient. Non-limiting examples NPL-binding compounds include: ZINC4096282, sialic acid alditol, MolPort-005-972-726, MolPort-005-975-059, MolPort-000-523-173, MolPort-004-939-783, ZINC27757562, ZINC102736687, ZINC8845752, ZINC8845753, ZINC68601727, ZINC102738233, ZINC67909210, ZINC4476643, ZINC4595517, ZINC1704299626, MolPort-003-875-380, MolPort-016-586-217 (ZINC4595517), or a pharmaceutically acceptable salt thereof (see, FIGS. 3A-3C).

The NPL-binding compound may be a compound illustrated in the following structure (i):

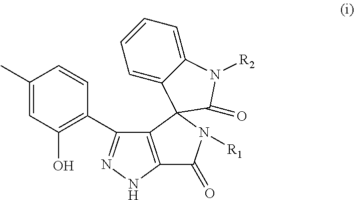

wherein,
   R₁ and R₂, are, independently, methyl, hydroxyl, or hydrogen, or a pharmaceutically-acceptable salt thereof. As such, R₁ and R₂ may be combined as indicated in Table 1.

TABLE 1
analogues of compound 173

| Compound | R₁ | R₂ |
|---|---|---|
| 173 | methyl (Me) | —Me |
| 173.1 | —Me | Hydroxyl (—OH) |
| 173.2 | —Me | Hydrogen (—H) |
| 173.3 | —OH | —Me |
| 173.4 | —OH | —OH |
| 173.5 | —OH | —H |
| 173.6 | —H | —Me |
| 173.7 | —H | —OH |
| 173.8 | —H | —H |

The NPL-binding compound may be a compound having the structure (ii):

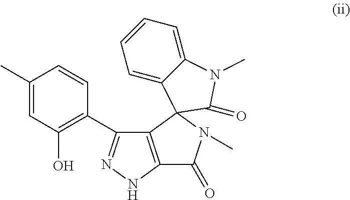

or a pharmaceutically-acceptable salt thereof, e.g., compound 173 (MolPort-000-523-173).

### Example 1

A network of genes mechanistically underlie the association of SNP rs12736689 with human chronotype based on genome wide association studies (GWAS). Post-GWAS functional genomic analysis were applied by employing ex vivo electrophoretic mobility shift assays (EMSA), SNP proteomic analysis, genomic HiC mapping analysis, and allele-imbalanced DNA pulldown-Western blots to determine allele-specific differences in transcription factor binding to rs12736689 and genomic interactions between rs12736689 and other DNA loci.

As a result, data demonstrates MYH9 and MYH10 transcription factor binding to rs12736689, which regulates the expression of neighboring genes RGS16 and non-neighboring gene NPL. As such, this SNPMYH9/10-RGS16-NPL axis may control chronotype behavior in the human population—with relevance to control of jet lag and other circadian behaviors in health and disease.

In further detail and in preliminary work, MYH9 binding to minor allele C suppresses the expression of RGS16, a G protein-signaling regulator, to decrease intracellular AC/cAMP levels. On the other hand, MYH10 binding to a C allele at rs12736689 activates a remote upstream gene, NPL, which encodes for an enzyme that degrades intracellular sialic acid. Sialic acid-containing molecules play a pivotal role in mediating signaling, immunological response, and cell-cell interaction. At the molecular level, via gain-of-function and loss-of-function analyses in cultured cells, substantial progress was made in demonstrating the causative circadian biology of the MYH-9/10-rs12736689-NPL axis. To summarize, it was found that this fSNP alters the binding of MYH-10 to modulate the expression of two neighboring genes (RNAseL, RGS16) and one non-neighboring gene (NPL). Furthermore, dysregulation of MYH-9 and MYH-10 alters cellular circadian rhythms in period and amplitude in vitro. These findings indicate that the circadian activity of MYH-10 is due, at least in part, to altered expression of NPL and potentially its control of sialic acid.

In cultured cells, it found that NPL is down-regulated by hypoxia (data not shown), consistent with population-level data of a chronotype shift to an earlier time point in hypoxic diseases. Supporting the role of NPL in hypoxic diseases of the vasculature, it was found (data not shown) that NPL deficiency promotes endothelial cell apoptosis and reduction of endothelial proliferation and angiogenesis-both cellular pathophenotypes seen in a prototypical hypoxic vascular disease, pulmonary hypertension. Therefore, the data support a model by which NPL plays a central role in controlling how rs12736689 and hypoxia control chronotype shifts, and this may depend upon sialic acid production.

To define small molecules that may control the function of these molecular axis factors, druggability simulations were performed using probe compounds that contain drug-like functional groups and/or features shared by potential lead compounds. The most important binding pockets were identified with evaluation of binding poses of probes, and pharmacophore modeling, using Pharmmaker. Virtual screening was performed of pharmacophore models against libraries of small compounds which are docked onto the target using the Pharmit server. A ranked list of small molecule compounds predicted to bind and control activity of NPL was generated. These molecules are tested for predicted activity to control circadian rhythm and jet lag in cell culture and animal models of jet lag. Priority U.S. Provisional Patent Application No. 63/374,456 filed Sep. 2, 2022, the disclosure of which is hereby incorporated herein by reference in its entirety, describes preliminary work related to identification of compounds that bind NPL, and which are expected to.

The primary role of NPL (Neuraminidase, N-Acetylneuraminic Acid Lyase) is to catalyze the cleavage of N-acetylneuraminic acid (sialic acid) into pyruvate and N-acetylmannosamine. Sialic acids form a diverse family of negatively charged monosaccharides with varying structures. Presently, there is no experimentally resolved structure available for human NPL (NPL_HUMAN; Q9BXD5). In our study, we utilized the machine learning tool AlphaFold2-generated structure. To construct pharmacophore models, we employed a druggability simulation methodology developed in the Bahar lab, along with docking simulations.

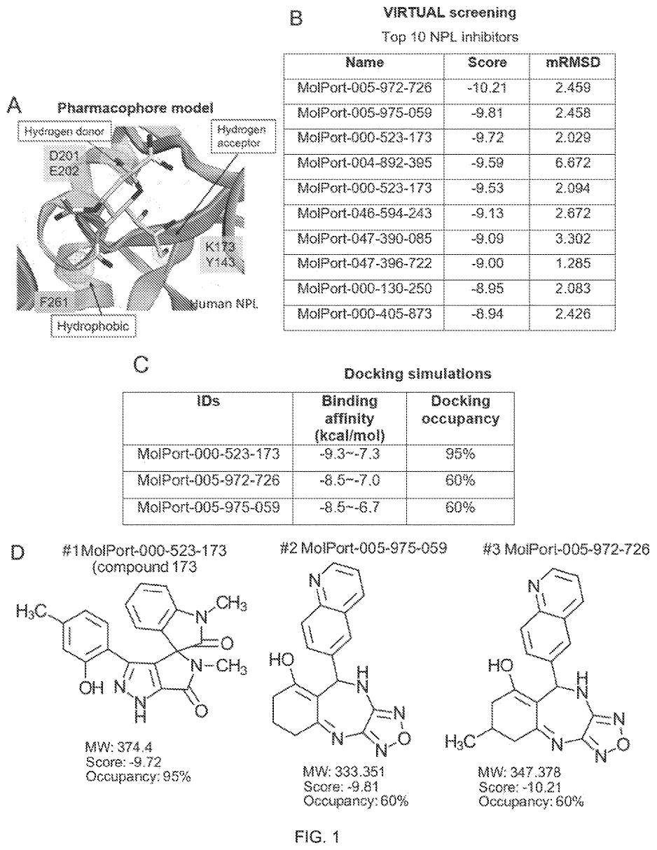

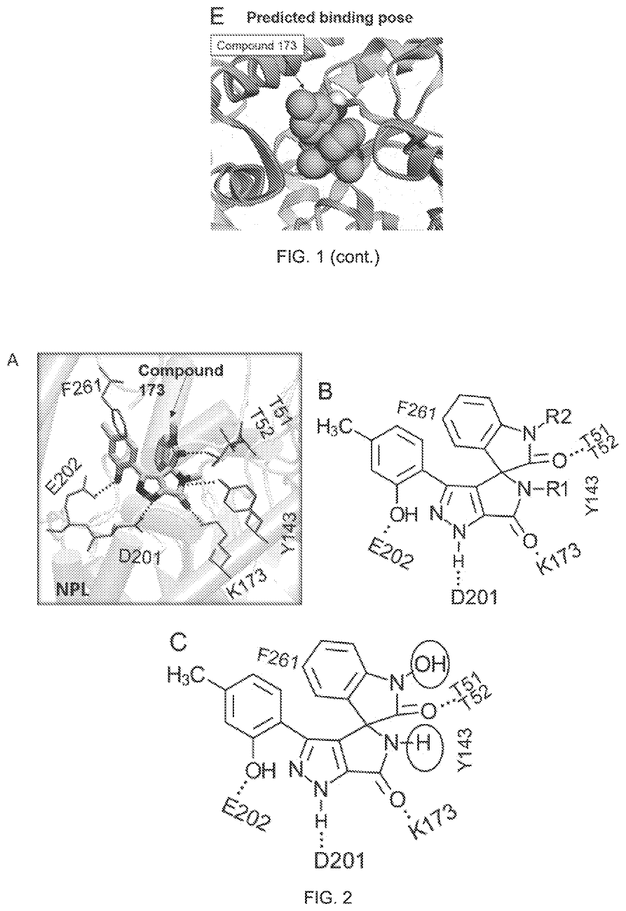

The employed pharmacophore model included a hydrogen donor located near two acidic residues D201 and E202 that are unfavorable for sialic acid binding, a hydrogen acceptor near the active site K173, and a hydrophobic feature near F261 (a specific residue of human NPL) (refer to FIG. 1 (A)). With this developed pharmacophore model, we screened compounds from the MolPort database using Pharmit. The top 10 NPL inhibitors, listed in FIG. 1 (B), were ranked based on their computed binding affinities (‘Score’ column). To cross-validate our predictions, we conducted additional docking simulations for each predicted inhibitor (FIG. 1 (C)). We introduced a new criterion called “docking occupancy,” which signifies the likelihood of a compound to attach itself to the designated target location. Elevated values of docking occupancy correspond to more favorable binding outcomes. Using this criterion, we re-ranked the predicted NPL inhibitors. By combining docking occupancy and binding affinities as criteria, we identified the top NPL inhibitor compound 173 (FIG. 1 (D)), which demonstrated both high affinity and docking occupancy. The predicted binding pose for compound 173 is shown in FIG. 1 (E).

In order to determine functional groups in lead compound 173 that facilitate its binding to NPL, multiple independent runs of molecular dynamics (MD) simulations were conducted. Through these MD simulations, the intermittent formation of five hydrogen bonds were observed during the relaxation of the protein structure (FIG. 2 (A)). Those hydrogen bonds observed during the MD simulations exhibited intimate interactions with the functionally essential residues of NPL: K173 (active site), Y143 (proton transfer site), and T51-T52 (substrate binding site). Moreover, these interactions were further strengthened by engaging with two acidic residues D201 and E202, emphasizing their significance in facilitating the binding process. By analyzing the binding poses of compound 173 to NPL during MD simulation courses, a generic structure for an NPL inhibitor was derived (FIG. 2 (B), and above). The functional groups (termed R₁ and R₂ in FIG. 2 (B)) may be fine-tuned to further enhance the binding of compound-173-like analogues. In examples, R₁ is methyl or H, and R₂ is methyl or OH.

### Example 2

Cell Culture. Primary human pulmonary artery endothelial cells (HPAECs) or mouse fibroblasts harboring a luciferase reporter under the control of a Period 2 (PER2) promoter (MF-Luc) were seeded into tissue culture dishes (Perkin Elmer) in a humidified incubator at 37 °C and 5% CO₂. For knockdown experiments, cells were pre-treated with 20 nM “Silencer Select” scrambled silencing RNA (siNC) or with antisense targeting of Np/(siNPL) for 48 hours (ThermoFisher). For chemical treatments, cells were treated with vehicle (DMSO) or 5 mM 173.

Apoptosis. Apoptosis was measured using the Caspase-Glo 3/7 Assay System (Promega) according to the manufacturer's instructions.

Cell Proliferation. Cell proliferation was measured using the BrdU Cell Proliferation Assay Kit (Cell Signaling Technologies) according to the manufacturer's instructions.

Lumicycle Recording. Prior to circadian measurement, cells were synchronized with 10 mM forskolin (Sigma) in Dulbecco's Modified Eagle Medium (DMEM) supplemented with 0.1% fetal bovine serum, and 100 mM Beetle luciferin (Promega) luciferase substrate was added for bioluminescent recording. Culture dishes were then sealed with sterile vacuum grease and placed in the Lumicycle 96 (Actimetrics) in a dehumidified incubator at 37 °C. Luminescence was recorded continuously for 7 days, and the initial 12 hours of data was omitted from the analysis to allow time for cell synchronization. Raw data were pre-processed for background correction and then fit to a sine curve with exponential decay of the form y=A sin([x−x₀]ω)e^{-x/τ}, where y represents the corrected luminescent signal, x represents time, A represents amplitude, x₀ represents phase shift, w represents frequency, and t represents the time decay constant. Period was calculated as 2p/w. Period was analyzed if the R² was greater than 0.8.

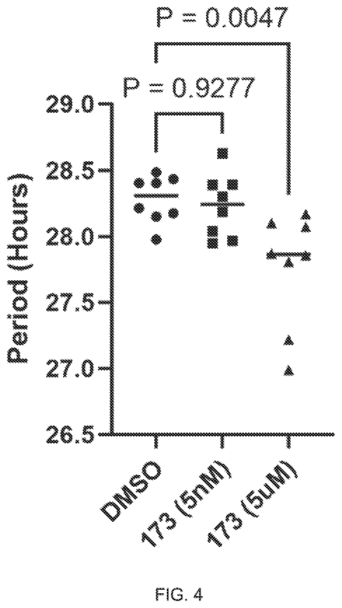

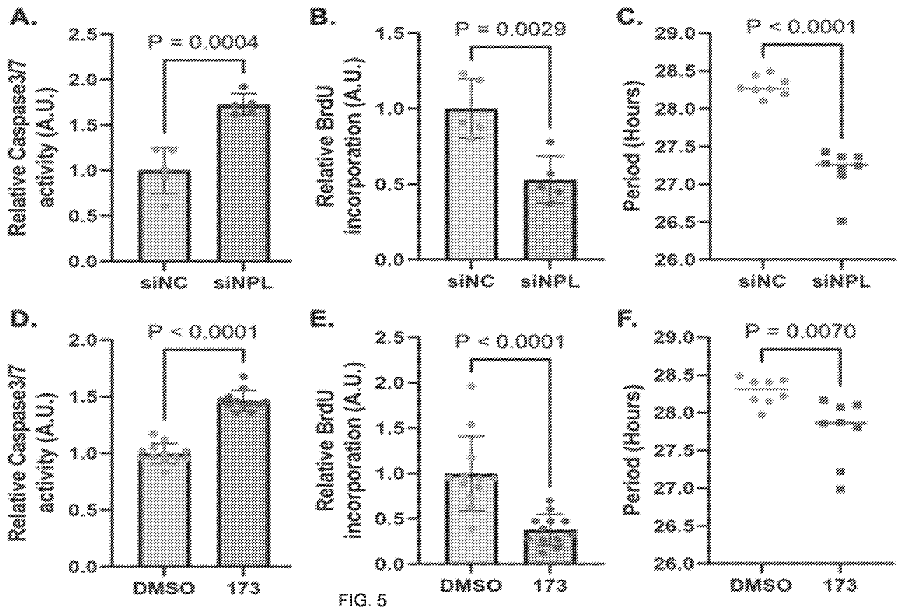

Compound 173 was shown to phenocopy siNPL in reducing circadian periodicity (FIGS. 4 and 5), establishing proof-of-concept that NPL-binding compounds can affect circadian rhythm. As shown in FIG. 5, compound 173 phenocopies siNPL. Apoptosis (FIG. 5, panels A & D) was increased and proliferation (FIGS. 5, B & E) was decreased in human pulmonary artery endothelial cells (PAECs) treated with siNPL or compound 173. Circadian period length was decreased in MF-Luc cells treated with siNPL or compound 173 (FIG. 5, panels C & F). A.U.: arbitrary units.

It will be readily appreciated by those skilled in the art that modifications may be made to the invention without departing from the concepts disclosed in the foregoing description. Accordingly, the particular embodiments described in detail herein are illustrative only and are not limiting to the scope of the invention, which is to be given the full breadth of the appended claims and any and all equivalents thereof.

1. A method of reducing jet lag, modifying chronotype, and/or or modulating a circadian clock of a patient, comprising: administering to the patient a compound that binds NPL, RGS16, MYH9, and/or MYH10 and/or modulates interaction of MYH9 and/or MYH10 with an endogenous nucleic acid sequence comprising rs12736689 in an amount effective to reduce jet lag or to modulate the circadian clock of the patient.

2. The method of claim 1, comprising administering a compound that binds NPL to the patient in an amount effective to reduce jet lag or to modulate the circadian clock of the patient.

3. The method of claim 2, wherein the compound that binds NPL is selected from the group consisting of: ZINC4096282, sialic acid alditol, MolPort-005-972-726, MolPort-005-975-059, MolPort-000-523-173, MolPort-004-939-783, ZINC27757562, ZINC102736687, ZINC8845752, ZINC8845753, ZINC68601727, ZINC102738233, ZINC67909210, ZINC4476643, ZINC4595517, ZINC1704299626, MolPort-003-875-380, MolPort-016-586-217, or a pharmaceutically acceptable salt thereof.

4. The method of claim 2, wherein the compound has the structure (i): 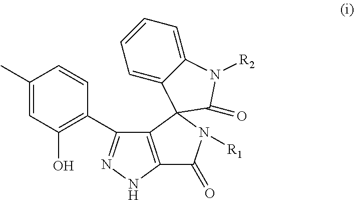
wherein,
R₁ and R₂, are, independently, methyl, hydroxyl, or hydrogen, or a pharmaceutically-acceptable salt thereof.

5. The method of claim 4, wherein R₁ is Me or H.

6. (canceled)

7. The method of claim 4, wherein R₂ is Me or OH.

8. (canceled)

9. The method of claim 4, wherein R₁ is H and R₂ is OH.

10. The method of claim 2, wherein the compound has the structure (ii): 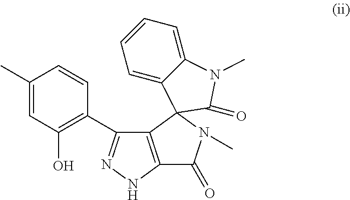
or a pharmaceutically-acceptable salt thereof.

11. A compound having the structure (i): 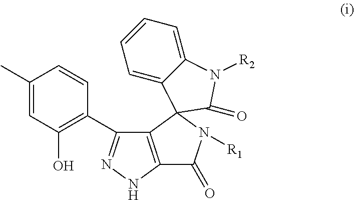
wherein,
R₁ and R₂, are, independently, methyl, hydroxyl, or hydrogen, and R₁ and R₂ are not both methyl,
or a pharmaceutically-acceptable salt thereof.

12. The compound of claim 11, wherein R₁ is Me or H.

13. (canceled)

14. The compound of claim 11, wherein R₂ is Me or OH.

15. (canceled)

16. The compound of claim 11, wherein R₁ is H and R₂ is OH.

17. The compound of claim 11, wherein the compound has the structure (ii): 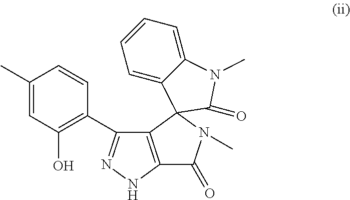
or a pharmaceutically-acceptable salt thereof.

18. A composition comprising:
a compound having the structure (i): 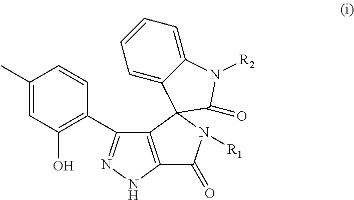
wherein,
R₁ and R₂, are, independently, methyl, hydroxyl, or hydrogen, and wherein R₁ and R₂ of the compound are not both methyl,
or a pharmaceutically-acceptable salt thereof in an amount effective to reduce jet lag, modify chronotype, and/or modulate a circadian clock of a patient; and
a pharmaceutically-acceptable excipient.

19. (canceled)

20. The composition of claim 18, wherein R₁ of the compound Me or H.

21. (canceled)

22. The composition of claim 18, wherein R₂ of the compound is Me or OH.

23. (canceled)

24. The composition of claim 18, wherein R₁ of the compound is H and R₂ of the compound is OH.

25. The composition of claim 18, wherein the compound has the structure (ii): 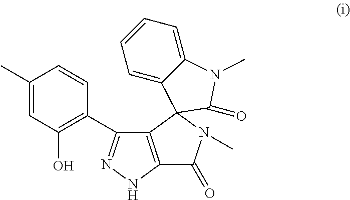
or a pharmaceutically-acceptable salt thereof.

26. A parenteral, topical, oral, transdermal, or transmucosal dosage form comprising the composition of claim 18.
